(19) United States
(12) Patent Application Publication
(10) Pub. No.: US 2022/0106667 A1
(43) Pub. Date: Apr. 7, 2022

(54) Integrated Acceleration of Algae and Microbial Screening Method and Facility for Recovery of Heavy Metals and Rare Earth Elements

(71) Applicant: Royal Biotech Inc, Beverly Hills, CA (US)

(72) Inventors: Philip Huang, Westlake Village, CA (US); E-Ray Huang, Thousand Oaks, CA (US)

(21) Appl. No.: 17/492,201

(22) Filed: Oct. 1, 2021

Related U.S. Application Data
(60) Provisional application No. 63/086,532, filed on Oct. 1, 2020.

(51) Int. Cl. C22B 59/00; C22B 7/00

(52) U.S. Cl. CPC C22B 59/00; C22B 7/006

(57) ABSTRACT

Provided are methods, systems, and facilities for screening, purification, and recovery of specific heavy metals and/or rare earth elements (REEs) from input materials including low-grade mines, tailings, sludge, rare earth, silt, and specific elements of Waste Electrical and Electronic Equipment (WEEE) by means of efficient microbial and/or algae screening method. The system and method of algae and microbial screening addresses the main problem of inefficient screening speed in the method of algae and microbial screening for recovery of specific heavy metals and/or REEs, which is too slow and time-consuming by integrated acceleration of the cultivation and screening of microbial and algae species of up to 50 times faster than current efficiencies by the application of a recovery rate metric model.

### CROSS REFERENCE TO RELATED APPLICATIONS

The current application claims benefit of U.S. Provisional Patent Application 63/086,532 filed Oct. 1, 2020.

### FIELD OF THE INVENTION

The present invention generally relates to the screening of heavy metals and rare earth elements (REE). More specifically, the present invention relates to a method and system for algae and microbial screening for recovery of heavy metals and REEs, and in particular for improving the speed of algae and microbial screening for recovery of heavy metals and REEs.

### BACKGROUND OF THE INVENTION

Rare earth elements (REEs) are a group of metals that have played a dominant role in technological progress and development of traditional industries. They are used in many everyday devices, including computer memory, DVDs, rechargeable batteries, cell phones, catalytic converters, magnets, and fluorescent lighting. REEs are also widely used in the military, metallurgic, petrochemical, glass-ceramic, agriculture, and new materials domains. Current heavy metal and/or REE screening methods have been applied for more than a hundred years with little change and improvement. Only the equipment manufacturing processes and pharmaceutical formulations have been improved, if at all, but there have been no innovations or breakthroughs in principles. Also, the distribution of REEs such as Sc, Y, La, Ce, in the earth's crust is quite scattered, and only a few REEs are concentrated in deposits that allow commercial exploitation. Rare earth is a mixture of many elements, and it is quite difficult to separate each element. Rare earth element (REE) screening causes great environmental pollution and health hazards particularly due to the high toxicity. Thus, methods and facilities for improving the speed of screening rare earth elements (REEs) and/or heavy metals are in demand.

The traditional methods for screening of heavy metals and REEs in mines, sludge, tailing, and waste electrical and electronic equipment (WEEE) industries are roughly divided into a few categories that include, hydrometallurgy and thermal methods, flotation method, gravity method, magnetic separation method, electric separation method, chemical mineral processing method, among others. However, many of these approaches are fraught with the problems of pollution and as labor health hazards, due to factors such as dust, sulfide, cyanide residues, and wastewater. These harmful and toxic waste materials may enter the food chains through soil and into plants and animals through which upon their respective consumption they enter the bodies of human beings. It is particularly difficult to fully recover heavy metals and/or REEs from low-grade mining, sludge, and tailing using the aforementioned current screening methods, some of which require consumables or chemical agents, and consume substantial quantities of energy and water.

Thus, the conventional screening methods to recover the desired and specific heavy metals and REEs have a number of problems associated with them including, environmental pollution, labor health hazards, excessive consumption of energy and water, difficulty in fully recovering from the low-grade input materials, requirement for costly consumables and/or chemical agents, and slow screening speed. Therefore, there is a need to find methods and facilities for screening heavy metals and REEs that reduce environmental pollution, labor health hazards, while saving energy and water resources, and provide greener and more efficient screening.

There have been an interest in developing biological methods for screening of heavy metals and/or REEs. A U.S. Pat. No. 7,837,760B2 provides a process to increase the bioleaching speed of ores or concentrates in an ore bed which is in the form of heaps, tailing dams, dumps, and other on-site bioleaching operations of sulfide metal species, the process comprising: inoculating the ore or concentrate to be bioleached with an inoculating solution containing isolated microorganisms of the Acidithiobacillus thiooxidans type, or together with isolated microorganisms of the Acidithiobacillus ferrooxidans type, with a total concentration of isolated microorganisms of about 1×107 cells/ml up to about 5×10 cells/ml, and wherein the bioleaching is carried out with or without the presence of native microorganisms that grow in the inoculating solution or of oxidizing ions; and carrying out the inoculation of the ore or concentrate until self-sustaining conditions of bacterial activity in the ore are reached, wherein self-sustaining conditions are reached when the bacterial count and iron-oxidizing activity of the bacteria in an effluent solution collected from the ore bed is similar in magnitude and composition to the bacterial count and iron-oxidizing activity of the bacteria in the inoculating solution. Another U.S. Ser. No. 10/501,822B2 provides a process of isolating or enriching a heavy metal present in a suspension containing a particulate mineral ore containing a heavy metal, comprising a step of incubating a suspension containing (i) a particulate mineral ore containing a heavy metal and (ii) biomass comprising a bacterium capable of binding the heavy metal; a step of separating the biomass having bound heavy metal from the suspension of the previous step; and a step of isolating the heavy metal from the biomass separated in the previous step; wherein said bacterium is selected from the following genera and species: Pseudochrobactrum, Bacillus pumilus, and Stenotrophomonas or from combinations thereof; and wherein the heavy metal is selected from ruthenium, rhodium, palladium, silver, osmium, iridium, platinum, gold, and/or rare earth metals. A European Patent, EP2813585A1 provides a process of isolating or enriching a rare earth element (REE) or a group of REEs from a solution or dispersion containing said REE or said group of REEs, comprising the following steps: (i) preparing a mixture comprising said solution or dispersion and biomass comprising at least one organism selected from any one of the following organism classes: eubacteria, archaea, algae, and fungi, whereby the at least one organism is capable of adsorbing or accumulating said REE or said group of REEs; (ii) incubating said mixture of step (i) for allowing the adsorption or accumulation of said REE or said group of REEs by said biomass; (iii) separating the biomass having adsorbed or accumulated REE(s) from the mixture of step (ii); and (iv) isolating said REE or said group of REEs from said biomass separated in step (iii). Another U.S. Pat. No. 5,055,402 provides a method for removing metal ions from an aqueous medium containing gone or more metal ions in solution comprising: contacting the aqueous medium with a composition having immobilized microorganisms capable of binding metal ions wherein the composition is prepared by heating an insoluble material having said immobilized microorganisms at an elevated temperature in the range of about 300° C. to about 500° C., and for a selected period of time maintaining the contact for a period of time, sufficient to permit binding of at least one of the metal ions in the aqueous medium to the microorganisms immuobilized in the composition. While a US patent application, US20200048732A1 provides method of recovering a target metal from a pregnant aqueous solution containing the target metal, or of recovering a target metal, the method comprising: (a) optionally a dissolution step comprising dissolving the target metal from a solid feedstock material with a lixiviant to form a pregnant aqueous solution containing target metal ions; (b) a biosorption step comprising contacting a microorganism with the pregnant aqueous solution such that at least a portion of the target metal biosorb to the microorganism, wherein the microorganism becomes metal laden and the pregnant aqueous solution becomes a barren solution; (c) a separating step comprising substantially separating the metal laden microorganism from the barren solution; and (d) a recovery step comprising recovery of the target metal from the metal laden microorganism.

The abovementioned prior art references which are incorporated here by reference, although illustrate some methods of algae and microbiological based screening methods for heavy metals and REEs, that have been developed to solve some of the problems with other traditional methods discussed above, but they are limited in scope due to being slow and time-consuming with slow screening speeds, and with operating times that are often dozens or more times longer than the other methods. Thus, there is a need to develop a method and system for solving these problems for screening methods based on biological systems of algae and microbial screening.

The present invention addresses the above discussed problems associated with and/or otherwise improve on conventional screening methods, devices and facilities for heavy metals and REEs recovery so as to maintain and promote the advantages of microbial and/or algae based screening methods and solve the above mentioned defects of microbial and/or algae based screening methods through an innovative screening method and system that are designed to provide a convenient and effective means of screening heavy metals and REEs while incorporating other problem-solving features.

### SUMMARY OF THE INVENTION

Generally, the present invention provides a method for screening of heavy metals and/or REEs where the underlying principle involves using algae and microbial extracellular and intracellular digestion to overcome the problems associated with other conventional screening methods for heavy metals and/or REEs, where the method and design of the present invention is applied for the number of input materials to stimulate the screening speed of algae and microorganisms. The method and system of the present invention combines various categories of algae and/or microbes which are cross-applied to enhance the screening efficiency and speed of algae and microbial screening for recovery of heavy metals and REEs, and comprises: (i) secretions from specific microbes or algae (A) that decompose specific heavy metals (X) into ions (other non-specific heavy metals are precipitated), and the specific heavy metals or REEs (X) ions are adsorbed on the surface of the A algae & microorganism to be recovered, (ii) secretions from another specific alternative algae or microbes (B) with an aversion to specific heavy metals and/or REEs (Y), where the secretions repel specific heavy metals or REEs (Y) (dissolve to the other components of the material) and produce the precipitation of the heavy metal (Y) element, and (iii) specific algae to inhale heavy metals or REEs for digestion and absorption.

In one aspect of the present invention, it discloses a method of algae and microbial screening for recovery of specific heavy metals and/or rare earth elements (REEs), the method comprising the steps of: selecting specific algae or microbial species by screening for specific heavy metals or REEs to find a specific algae or microbial species and analyzing the optimal size of input materials for the adsorption or repulsion effect on the input material by the specific algae or microbial species; adjusting the index variables of various factors in an incubation pool referred to as Pool A to find the best growth conditions for the specific algae or microbial species involving collecting a sample from a sampling port for sampling and analysis by an information data and control center for the reproduction of the specific algae and microbial species by testing the sample for concentration or growth; modulating the environmental conditions in the incubation pool by adjusting micro-current or magnetic variables to stimulate algae or microbial species metabolism and increase secretion or absorption and verifying it by collecting a sample from the sampling port for sampling and analysis by the information data and control center; readjusting the index variables of various factors in the incubation pool to find the best solubility of the secretion of the input material and verifying it by collecting a sample from the sampling port for sampling and analysis by the information data and control center; adjusting the revolutions per minute referred to as rpm of stirred tanks or reactors or speed of shakers in the incubation pool in an interactive manner by coordinating through the information data and control center.

In another aspect of the present invention, it discloses a method for improving the speed of algae and microbial screening for recovery of specific heavy metals and/or rare earth elements (REEs), the method comprising the steps of: selecting specific algae and microbial species for specific heavy metal and/or REE and input materials; incubating said specific algae and microbial species in an incubation pool referred to as Pool A, comprising specific nutrients and selective agents to stimulate excitation viable algae and microbial species that show rapid growth; verifying the change in volume as a measure of said rapid growth of said excitation viable algae and microbial species; modulating the environmental conditions in the incubation pool to obtain specific excitation viable algae and microbial species; adding milled input materials to the incubation pool; recovering specific heavy metals and/or REEs from said input materials by algae and microbial screening using the excitation viable algae and microbial species by selecting from a group consisting of (i) fully grinding, diluting and decomposing the excitation viable algae and microbial species to obtain secretions from specific excitation viable algae and microbial species referred to as (A) which decompose specific heavy metals and/or REEs into ions and precipitate the specific heavy metals and/or REEs referred to as (X), or (ii) fully grinding, diluting and decomposing the excitation viable algae and microbial species to obtain secretions from specific excitation viable algae and microbial species referred to as (B) which repel specific heavy metals and/or REEs and produce the precipitation of the heavy metal and/or REEs referred to as (Y), or (iii) using specific excitation viable algae to precipitate specific heavy metals and/or REEs present in the input materials after being absorbed by algae and collecting said algae for drying and heating, and obtaining the specific heavy metals and/or REEs by centrifugal separation, or a combination thereof; sampling and monitoring continuously the specific excitation viable algae and microbial species by collecting samples from a sampling port; analyzing the collected samples for parameters of microbial secretion or algae absorption and modulating the environmental conditions in the incubation pool by an information data and control center by the application of a recovery rate metric model referred to as RRM to identify and select the most suitable specific algae and microbial species for a specific heavy metals and/or REEs in the input materials and for improving the speed of algae and microbial screening for recovery of said specific heavy metals and REEs.

In another aspect of the present invention, it discloses a system for the method for improving the speed of algae and microbial screening for recovery of specific heavy metals and rare earth elements (REEs), the system comprising: an incubation pool used as a microbial culture tank or algae incubator referred to as Pool A; a sampling port for sampling and analysis of microbial species and algae reproduction; and an information data and control center, wherein, the information data and control center comprises: collecting real-time information on the analysis of specific parameters comprising microbial concentration, secretion concentration, solubility of the microbial culture tank, the growth of microbial species and absorption of algae incubator; monitoring the amount of water, oxygen, carbon dioxide, nutrients, selective agents, temperature, pH value, light sources strength, micro-current, magnetic field, and sending the collected and monitored information to the control center, analyzing automatically and calculating the optimal recovery rate effect simulation for Pool A, and issuing the various ACTION commands for Pool A for said optimal recovery rate effect by the application of a recovery rate metric model referred to as RRM to identify and select the most suitable specific algae and microbial species for a specific heavy metals and/or REEs in the input materials and for improving the speed of algae and microbial screening for recovery of said specific heavy metals and REEs, and wherein the sampling port is connected to the information data and control center.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWING

The accompanying drawings are included to provide a further understanding of the invention, and are incorporated in and constitute a part of the present invention and, together with the description, serve to explain the principle of the invention.

In the drawings,

FIG. 1 is an illustration of the overall principle underlying the present invention.

FIG. 2 is an illustration of one embodiment of the microbial/algae growth model of the present invention.

FIG. 3 is an illustration of one embodiment of the microbial secretion model/algae digestion Model of the present invention.

FIG. 4 is an illustration of one embodiment of the microbial dissolution model/algae absorption Model of the present invention.

FIG. 5 is an illustration of an alternative embodiment of the present invention with the microbial/algae growth model, the microbial secretion model/algae digestion model, and the microbial dissolution model/algae absorption Model.

FIG. 6 is an illustration of one embodiment of the present invention with the microbial/algae growth model, the microbial secretion model/algae digestion model, and the microbial dissolution model/algae absorption model.

### DETAILED DESCRIPTION OF THE INVENTION

The present invention may be understood more readily by reference to the following detailed description of the invention taken in connection with the accompanying drawing figures, which forms a part of this disclosure. All illustrations of the drawings are to describe selected versions of the present invention and are not intended to limit the scope of the present invention. It is to be understood that this invention is not limited to the specific devices, systems, conditions or parameters described and/or shown herein and that the terminology used herein is for the example only, and is not intended to be limiting of the claimed invention.

Also, as used in the specification including the appended claims, the singular forms ‘a’, ‘an’, and ‘the’ include the plural, and references to a particular numerical value includes at least that particular value unless the content clearly directs otherwise. Ranges may be expressed herein as from ‘about’ or ‘approximately’ another particular value. When such a range is expressed it is another embodiment. Also, it will be understood that unless otherwise indicated, dimensions and material characteristics stated herein are by way of example rather than limitation, and are for better understanding of sample embodiment of suitable utility, and variations outside of the stated values may also be within the scope of the invention depending upon the particular application.

As used herein, input materials include mining, low-grade mining, sludge, tailing and waste electrical and electronic equipment (WEEE).

As used herein, algae and microbial screening means bioaccumulation, bioremediation, tolerance of specific algae and microbial species that absorb and metabolize, adsorb or repel specific heavy metals and/or REEs and can accordingly be used for algae and microbial screening and recovery of specific heavy metals and/or REEs by using their intracellular and extracellular digestion mechanisms. Algae screen included anti-microbial activities.

As used herein, algae means “a plant or plantlike organism of any of several phyla, divisions, or classes of chiefly aquatic usually chlorophyll-containing nonvascular organisms of polyphyletic origin that usually include the green, yellow-green, brown, and red algae in the eukaryotes and especially formerly the cyanobacteria in the prokaryotes” and not limited to for example, green algae—Enteromorpha intestinalis (Linnaeus) Nees, Cladophora glomerata (Linnaeus) Kutzing, etc.

As used herein, microbes, microorganisms, microbiological and microbial species have been used interchangeably and mean but are not limited to for example, Acidithiobacillus ferrooxidans, Sulfate-reducing bacteria CL4, Mycobacterium phlei, Bacilluspolymyxa, Micococcus luteus, etc.

Embodiments will now be described in details with reference to the accompanying drawings. To avoid unnecessarily obscuring in the present disclosure, well-known features may not be described, or substantially the same elements may not be redundantly described, for example. This is for ease of understanding. The drawings and the following description are provided to enable those skilled in the art to fully understand the present disclosure and are in no way intended to limit the scope of the present disclosure as set forth in the appended claims.

The biggest problem of the algae and microbiological screening method in use for heavy metals and/or REEs is the screening speed, which is too slow and time-consuming, which leads to the recovery of the same volumes of input materials. The required operating time is often dozens or more times than other methods. The present invention provides a method that may be applied to many input materials to accelerate screening by up to 50 times through the cultivation of algae and microorganisms. The present invention employs the principle of microbial/algae metabolism, based on the adsorption of specific microbes/algae (A) to specific heavy metals/REEs (X) and the aversion of alternative microbes/algae (B) to specific heavy metals/REEs (X). Two designs are cross-applied to enhance screening efficiency.

Compared with other existing methods and facilities, the present invention produces 90% savings of energy and water without requiring consumables or chemical agents. The application range of the present invention is wide: screening and purification of low-grade mines, tailings, rare earth, and even silt as well as specific elements of waste electrical and electronic equipment (WEEE).

The present invention discloses a method and system of using specific algae or microorganisms to secrete some substances (e.g., polysaccharides, esters, proteins) and release them into the environment, changing environmental conditions, and so forth to produce specific heavy metal/or REE precipitation and recover these heavy metals/REEs, as can be described in Case 1 (uses of Acidithiobacillus ferrooxidans to secrete hydrogen sulfide, precipitating copper) and Case 2 (use of sulfate-reducing bacteria CL4 to release glycerin, precipitating zinc).

There are several principles of algae & microbial screening. The invention here is based on the principle of algae and microbial metabolism, following the adsorption of specific microbes and/or algae (A) to specific heavy metals and/or REE(X) and the aversion of alternative microbes and/or algae (B) to the specific heavy metals and/or REE (X). Two designs are cross applied to complete the screening efficiency. The extracellular digestion of different algae and microorganisms is used to repel or adsorb specific metal elements/or REE, resulting in the precipitation of specific heavy metals and/or REE. Two different algae and microbial secretions (A and B) are fully ground, and then diluted and decomposed separately. The secretion decomposes specific heavy metals and/or REE (X) into ions (other non-specific heavy metals and/or REE are precipitated), but the specific heavy metal and/or REE ions are adsorbed on the surface of the A algae and microorganism to be recovered. Alternatively, B secretions repel specific heavy metal elements and/or REEs (Y) (dissolve to the other components of the material) produce the precipitation of the heavy metal and/or REEs (Y). Another alternate means is where specific algae inhale heavy metals and/or REE for digestion and absorption. The order of steps and processes disclosed herein can be adjusted to reflect differences between various algae and microbial species and the heavy metal and/or REE targeted for recovery.

In accordance with one embodiment of the present invention, it discloses a method of algae and microbial screening for recovery of specific heavy metals and/or rare earth elements (REEs), the method comprising the steps of: selecting specific algae or microbial species by screening for specific heavy metals or REEs to find a specific algae or microbial species and analyzing the optimal size of input materials for the adsorption or repulsion effect on the input material by the specific algae or microbial species; adjusting the index variables of various factors in an incubation pool referred to as Pool A to find the best growth conditions for the specific algae or microbial species involving collecting a sample from a sampling port for sampling and analysis by an information data and control center for the reproduction of the specific algae and microbial species by testing the sample for concentration or growth; modulating the environmental conditions in the incubation pool by adjusting micro-current or magnetic variables to stimulate algae or microbial species metabolism and increase secretion or absorption and verifying it by collecting a sample from the sampling port for sampling and analysis by the information data and control center; readjusting the index variables of various factors in the incubation pool to find the best solubility of the secretion of the input material and verifying it by collecting a sample from the sampling port for sampling and analysis by the information data and control center; adjusting the revolutions per minute referred to as rpm of stirred tanks or reactors or speed of shakers in the incubation pool in an interactive manner by coordinating through the information data and control center.

In another embodiment of the present invention, it discloses a method of algae and microbial screening for recovery of specific heavy metals and/or rare earth elements (REEs) according to the present invention, wherein the adsorption or repulsion effect on the input material by the specific algae or microbial species consists of: (i) fully grinding, diluting and decomposing the excitation viable algae and microbial species to obtain secretions from specific excitation viable algae and microbial species referred to as (A) which decompose specific heavy metals and/or REEs into ions and precipitate the specific heavy metals and/or REEs referred to as (X), or (ii) fully grinding, diluting and decomposing the excitation viable algae and microbial species to obtain secretions from specific excitation viable algae and microbial species referred to as (B) which repel specific heavy metals and/or REEs and produce the precipitation of the heavy metal and/or REEs referred to as (Y).

In another embodiment of the present invention, it discloses a method of algae and microbial screening for recovery of specific heavy metals and/or rare earth elements (REEs) according to the present invention, wherein the various factors consist of external and internal factors, wherein the external factors comprise temperature, light, pH value, oxygen, carbon dioxide, amount of water, and wherein the internal factors comprise nutrients, selective agents, ionic strength, polarity.

In another embodiment of the present invention, it discloses a method of algae and microbial screening for recovery of specific heavy metals and/or rare earth elements (REEs) according to the present invention, wherein the information data and control center can adjust the index variables of various factors in the incubation pool to shift the mode of operation of the incubation pool in an outcome selected from a group consisting of growth mode, secretion mode, dissolution mode, recovery rate measurement mode, or a combination thereof, for recovery of specific heavy metals and/or REEs.

The efficiency screening method of microorganisms & algae has been paid attention shortly because of the following advantages:

i. Highly environmental protection, low environmental pollution (without chemical residues).
ii. The high specificity of screening and recovery concentrates (can be screened for specific heavy metal/or REE types, reducing the multi-element mixing of screening concentrates, improving the grade and value of concentrate concentrates).
iii. Low screening cost.
iv. Energy and water savings, where compared with other existing methods and facilities, this innovative method usually saves 9/10 of energy and water consumption.
v. There is no need for consumables or chemical agents, where this method may save tens of thousands of dollars even more in electricity, water, or chemical reagents per day. It also does not cause environmental pollution problems, such as wastewater or air pollution. There are no concerns about sulfide, cyanide residues, etc. that are labor health hazards. Furthermore, related algae & microbial vectors can be repeatedly and automatically generated.
vi. High recovery rate (Esp. for low-grade mine & tailing)—Recovery % represents the ratio of the weight of metal or mineral values recovered in the concentrate to 100% of the same constituent in the heads or feed to the process, expressed as a percentage. It may be calculated in several different ways, depending on the data available.

The present invention provides a method and system for algae and microbiological screening that not only can increase the recovery rate of related specific elements and reduce the impurity content, but it can also create at least a 20% to 200% increase in income and reduce considerably the enormous initial construction cost of the screening base (for example, the required land area is reduced around by 70%, eliminating the need to invest in gravity table, and so forth).

Precision screening is based on the high efficiency of high recovery of specific heavy metals or elements contained in mines (or tailings, sludge, soil) or wastes electrical and electronic equipment (WEEE) to reduce “residue & screening losses”. How to improve the screening recovery rate, create “precision screening” to improve the efficiency of refining (reduce refining cost, improve the quality of materials, upgrade environmental protection and less cost . . . etc.)? It is currently a screening problem for mines, tailings, sludge, and waste electrical and electronic equipment (WEEE) (especially in the recycling industry). To solve this problem, of course, one must first be able to check the properties of the input material in real-time on the spot, and quick feedback to facilitate the timely adjustment of the front-end or mid-end and back-end core related processes. If one uses microbial screening methods for heavy metal particles, in addition to the selection of microbial species, the concentration of microorganism content, the speed of microorganism screening, and the sequence and configuration of microorganism screening will be one of the key factors that affect the efficiency of screening recovery. Thus, regardless of whether the principle of recovery is to use algae and microorganisms to repel or adsorb specific heavy metals and/or REEs, how should the screening speed of algae and microorganisms be stimulated remains a problem? For example: use of specific algae and microorganisms to secrete some substances (for example polysaccharides, esters, proteins, etc. . . . ) and their release into the environment, and change in the environmental conditions, etc., produces specific heavy metal and/or REE precipitation, that can then be beneficially recovered.

The present invention provides a method comprising the following steps as explained below:

Selection of algae and microbiological species for specific heavy metal/or REE and input materials. The application of a real-time video electron microscope for observation and records analyzes algae or microorganism adsorption or digestion performance. Then make full use of genetic engineering (for example CRISPR-cas12, CRISPR-cas9, CRISPR-cas13 . . . ) to edit the most suitable algae & microorganisms.

Incubation of algae and microbiological species—excitation viable microorganisms or algae—rapid growth.

According to the metabolic function of specific algae & microbial species, the optimal growth environment conditions are designed according to the following factors:

i. Nutrients
ii. Selective Agents (Selective media allow certain types of organisms to grow and inhibit the growth of other organisms . . . )
iii. Oxygen
iv. Temperature

### v. pH Value

vi. Water Ability
vii. CO2
viii. Light (Photosynthesis)

Adding paraffin oil into the micro bio incubating pool, to cover the surface of the pool avoids direct contact of the context of the pool with air. The Royal Biotech's specific nutrients & selective agents can increase at least 5 times high performance than traditional nutrients & selective agents.

In the meantime, the changed volumes of microorganisms & algae should be verified. For example, through the color changed method & time calculation to verify the volumes of microorganisms.

Increase the secretion of secretions of the microorganism or intensify algae growth—by introducing microcurrent or magnetic force into one direction, it can be introduced into the algae & microorganisms to stimulate the electromagnetic field of the algae & microorganisms, to promote the metabolism in the cells of the algae & microorganisms and increase the amount of secretion or growth. On the contrary, it is also possible to use micro electric current or concentrate the magnetic force in one direction and introduce it into the environment where the algae & microorganisms are located, so that the algae & microorganisms have a disordered or damaged activity mechanism, and then lose their activity force.

i. Establish a protective shield against magnetic field & telecommunications interference from the outside of the pool.
ii. Install measuring magnetic field and current counter link to the pool.
iii. Install secretion measurement scale around the pool.
Further, use of centrifugal principle to separate unnecessary components when necessary is made.

Improve the speed of secretion dissolution of microorganism or enhance the speed of algae digestion and absorption:

Use microbial secretions to precipitate specific heavy metal elements/or REE in the input material where there is the application of diffusion principle, where high concentration to low concentration diffusion means that the concentration of the secreted solvent is higher than the specific metal concentration of the input material. It is difficult to dissolve specific metal and/or/REE into secretion solvent. On the contrary, high concentrations of specific medium metal elements/or REE can penetrate a low concentration of the secreted solvent.

Consider the elements within the input traits:

i. Ionic strength (a measurement of ion concentration in solution)
ii. Polarity

The uses of algae to precipitate specific heavy metal elements/REE in the input material after being absorbed by algae. Then collect the algae for drying & heating. The required heavy metal elements and/or REE will be obtained by centrifugal separation. For reaching the above target, design & adjust environmental factors to control:

i. pH
ii. Temperature
iii. Pressure (gas)
iv. Sunlight
v. Solvent,
vi. even consider Salinity

To the best dissolving or absorption environment conditions, when necessary, take mechanically stir in the stirred tanks or reactors for input materials' bioleaching or absorption.

Then help to increase the rate of dissolution of secretions or digestion & absorption ability of algae.

From the above three aspects, accelerate the quantitative screening of input materials by microorganisms or algae.

In accordance with one embodiment of the present invention, it discloses a method for improving the speed of algae and microbial screening for recovery of specific heavy metals and/or rare earth elements (REEs), the method comprising the steps of: selecting specific algae and microbial species for specific heavy metal and/or REE and input materials; incubating said specific algae and microbial species in an incubation pool referred to as Pool A, comprising specific nutrients and selective agents to stimulate excitation viable algae and microbial species that show rapid growth; verifying the change in volume as a measure of said rapid growth of said excitation viable algae and microbial species; modulating the environmental conditions in the incubation pool to obtain specific excitation viable algae and microbial species; adding milled input materials to the incubation pool; recovering specific heavy metals and/or REEs from said input materials by algae and microbial screening using the excitation viable algae and microbial species by selecting from a group consisting of (i) fully grinding, diluting and decomposing the excitation viable algae and microbial species to obtain secretions from specific excitation viable algae and microbial species referred to as (A) which decompose specific heavy metals and/or REEs into ions and precipitate the specific heavy metals and/or REEs referred to as (X), or (ii) fully grinding, diluting and decomposing the excitation viable algae and microbial species to obtain secretions from specific excitation viable algae and microbial species referred to as (B) which repel specific heavy metals and/or REEs and produce the precipitation of the heavy metal and/or REEs referred to as (Y), or (iii) using specific excitation viable algae to precipitate specific heavy metals and/or REEs present in the input materials after being absorbed by algae and collecting said algae for drying and heating, and obtaining the specific heavy metals and/or REEs by centrifugal separation, or a combination thereof; sampling and monitoring continuously the specific excitation viable algae and microbial species by collecting samples from a sampling port; analyzing the collected samples for parameters of microbial secretion or algae absorption and modulating the environmental conditions in the incubation pool by an information data and control center by the application of a recovery rate metric model referred to as RRM to identify and select the most suitable specific algae and microbial species for a specific heavy metals and/or REEs in the input materials and for improving the speed of algae and microbial screening for recovery of said specific heavy metals and REEs.

In another embodiment of the present invention, it discloses a method for improving the speed of algae and microbial screening for recovery of specific heavy metals and/or rare earth elements (REEs) according to the present invention, wherein the modulating the environmental conditions in the incubation pool to obtain specific excitation viable algae and microbial species results from selecting a mode of operation from a group consisting of intensifying algae growth or increasing the secretion of secretions from the microbial species or improving the speed of secretion dissolution of microbial species or enhancing the speed of algae digestion and absorption, or a combination thereof.

In another embodiment of the present invention, it discloses a method for improving the speed of algae and microbial screening for recovery of specific heavy metals and/or rare earth elements (REEs) according to the present invention, wherein the application of a recovery rate metric model referred to as RRM comprises recovery rate prediction, and adjusting various factors suitably for identification and selection of the most suitable specific algae and microbial species for a specific heavy metals and/or REEs in the input materials and for improving the speed of algae and microbial screening for recovery of said specific heavy metals and REEs, wherein the various factors consist of external and internal factors, wherein the external factors comprise temperature, light, pH value, oxygen, carbon dioxide, amount of water, and wherein the internal factors comprise nutrients, selective agents, ionic strength, polarity.

In another embodiment of the present invention, it discloses a method for improving the speed of algae and microbial screening for recovery of specific heavy metals and/or rare earth elements (REEs) according to the present invention, wherein the recovery rate metric model referred to as RRM is integrated into intelligent evolutionary learning platform involving machine learning run by the information data and control center and comprises one or more stochastic equations which are composed of variables and coefficients to identify and select the most suitable specific algae and microbial species for a specific heavy metals and/or REEs in the input materials and for improving the speed of algae and microbial screening for recovery of said specific heavy metals and REEs, wherein a set of minimums (m) most suitable marked algae or microbial species are identified along with influencing factors from high-dimensional (n) training samples to establish a mathematical prediction model.

In the present invention, faculties are provided as follows: Set up a system information data and control center: the data center collects real-time information on the analysis of specific microbial concentration, secretion concentration, solubility, etc. of the microbial culture tank or the growth, absorption of algae incubator. It also monitors the amount of water, oxygen, CO2, Nutrients, selective agents and temperature, pH Value/Light sources strength, micro-current, magnetic field, related information, send it to the control center. Automatic analysis and calculation of the POOL A, optimal recovery rate effect simulation, and then issue various ACTION commands.

The procedure can be enumerated as follows:

1) Design microbial culture tank or algae incubator: “POOL A”—
The following special pipelines are configured from the outside world to transport into POOL

### A:

Water pipe
Oxygen tube
Carbon dioxide pipe
Nutrient's tube
Input Selective agents and adjust pH Value, Light source, Temperature, etc.
2) Sampling port settings (for sampling and analysis of microbial or algae reproduction) and connect to the data center.
3) The data center will analyze the parameters of the status of microbial or algae reproduction as the base, perform computer simulation calculations, and calculates the best adjustment recommendations for various factors (water availability, carbon dioxide, oxygen, nutrients, Selective agents). Microbial varieties to create the best growing environment conditions for POOL A.
For an estimate of microbial volumes:
i. Semi-quantitative analysis of microbial content by color change method
ii. The color change method integrated with ELISA, Gene-probe, plate counting method to be higher specificity (99%) & sensitivity (reach to 1 CFU/ml), fast test response (5 times faster than a traditional method) called MBS® method
For an estimate of algae volumes by volume and weight scaler
4) Wait for specific microorganisms or algae to increase to a certain level of POOL A, add the appropriate amount of milled input materials, and at the same time introduce micro-current or magnetic force to stimulate the metabolism of microorganisms or algae and increase the % of secretion or absorption. This space is designed with a protective shield against magnetic field & telecommunication interference.
5) Set from the sampling port (sampling and analysis of microbial secretion or algae absorption) Connect to the data center.
6) The data center will analyze the parameters of microbial secretion or algae absorption as the base, perform computer simulation calculations, and calculate all kinds of factors (microcurrent or magnetic strength, etc.). Adjust recommendations that affect the best secretion or absorption. Then develop the most suitable for the specific microbial or algae metabolism function of good secretion concentration condition or absorption level.
7) In POOL A, specific microorganisms or algae are propagated and secreted to a certain concentration, and a large amount of “preparation of selected heavy metal and/or REE input materials” is introduced for decomposition and dissolution or absorption operations.
8) According to the optimal decomposition and melting speed-related parameters estimated by the data center in advance, it is provided to the POOL A control center, and the following environmental factor index adjustments are issued: pH Value, Temperature, (Pressure) . . . etc. And moderate stirring to catalyze the precipitation rate of specific heavy metals and/or REE.

In accordance with one embodiment of the present invention, it discloses a system for the method for improving the speed of algae and microbial screening for recovery of specific heavy metals and rare earth elements (REEs), the system comprising: an incubation pool used as a microbial culture tank or algae incubator referred to as Pool A; a sampling port for sampling and analysis of microbial species and algae reproduction; and an information data and control center, wherein, the information data and control center comprises: collecting real-time information on the analysis of specific parameters comprising microbial concentration, secretion concentration, solubility of the microbial culture tank, the growth of microbial species and absorption of algae incubator; monitoring the amount of water, oxygen, carbon dioxide, nutrients, selective agents, temperature, pH value, light sources strength, micro-current, magnetic field, and sending the collected and monitored information to the control center, analyzing automatically and calculating the optimal recovery rate effect simulation for Pool A, and issuing the various ACTION commands for Pool A for said optimal recovery rate effect by the application of a recovery rate metric model referred to as RRM to identify and select the most suitable specific algae and microbial species for a specific heavy metals and/or REEs in the input materials and for improving the speed of algae and microbial screening for recovery of said specific heavy metals and REEs, and wherein the sampling port is connected to the information data and control center.

In addition to invented speeding up the screening of microorganisms or algae (at least 50 times faster than the current microbial screen method), it is also possible to estimate the optimal parameter combination & recovery rate through the computing integrated with the recovery measurement (metric) model (Predictive model). This estimated recovery rate measurement model—Simulation calculation. It can save a lot of experiments trial and error (time, cost, etc.).

The key to the data & control center is our invented recovery rate metric model (called “RRM”) also. Recovery metric model integrated into the data & control center. Offer recovery rate prediction, then let the control center adjusts suitably factors for recovery of heavy metal and/or REE by microbiology or algae.

The metering and recover model include one or more stochastic equations, which succinctly and effectively describe and summarize the quantitative characteristics of a real recycling and screening system, and more profoundly reveal the quantity change rule of the recycling system. It is composed of systems of equations, which are composed of variables and coefficients. Among them, the system is also composed of equations.

The metering recovery model reveals the quantitative relationship between various factors in the screening activities and is described by a random mathematical equation. Integrate all the data into software program.

The flow can be summarized as going from fixed specific heavy metal and/or REE to selection of variety or groups of microbial species or algae to determination and regulation of factors that include light, temperature, water ability, oxygen, carbon dioxide, pH value), nutrients, selective agents, microcurrent, etc. leading to microbial or algae changes in terms of factors including variety selection, growth number, secretion, and/or solubility to finally determination and regulation of recovery rate changes coordinated for the specific heavy metal and/or REE as desired.

The discovery of the most suitable marked microbiology/or algae and the establishment of predictive models is to provide the application for recovery model of heavy metal/REE with microorganisms/or algae.

Statistical technology is concerned with causal reasoning and is often used for the discovery of the most suitable marked microbiology/or algae (included influencing factors); machine-learning emphasizes the prediction results and is suitable for identifying a group of most suitable marked microbiology/or algae (included influencing factors) and establishing mathematical prediction models. To identify a set of minimums (m) most suitable marked microbiology/or algae (included influencing factors) from high-dimensional (n) training samples to establish a mathematical prediction model and achieve the best prediction accuracy is a very challenging dual-objective combination optimization C (n, m) problem and evolutionary computing is the first choice for solving combinatorial optimization problems. When the number of training samples is not sufficient, it will cause the underdetermined problem of non-unique solutions. If labeling uncertainty occurs, for example, labeling samples may encounter CROSS TALK, which will reduce the accuracy of prediction. When faced with insufficient data and information coverage, microbiology or screening technology experts can provide expert knowledge to make up for it.

The intelligent evolutionary learning platform can introduce expert knowledge into evolutionary learning, consider the uncertainty of sample labeling, identify a set of robust most suitable marked microbiology/or algae (included influencing factors), and establish a mathematical prediction model. Making good use of the growing feedback mechanism of the data set, the evolutionary learning platform can gradually optimize the prediction model, identify a more correct set of most suitable marked microbiology/or algae (included influencing factors), and provide ranking analysis of most suitable marked microbiology/or algae according to the predicted contribution, as well as design optimization of input parameters and simulation results. For example, our evolutionary learning uses the divide and conquer technology of the intelligent evolution algorithm to solve the high-dimensional combination optimization problem and uses the inherited dual-target genetic algorithm to find and identify a set of most suitable marked microbiology/or algae features to identify the best. The semi-supervised learning method of the control group overcomes the problem of labeling uncertainty and uses embedded domain knowledge and evolutionary computing technology to overcome the under-determined problem of insufficient data.

The recovery rate metric model (RRM) lets technicians be easy to reach to best control for recovery rate, environmental request. Do not need too much tried & error, cost wasted.

Use microorganisms or algae to screen specific heavy metals/or REE:

First of all— screening for specific heavy metals/or REE to find the most suitable algae or microorganism species (also analyze the optimal size of input materials for the algae or microorganism's adsorption or repulsion effect on input material). Selection of species.
Second— adjust the index variables of various factors to find the best growth conditions for the species of algae or microorganisms (taking the sample to test concentration or growth).
Third— adjust micro-current or magnetic variables to stimulate algae or microbial metabolism and increase secretion (taking the sample to check secretion increase) or absorption.
Fourth—adjust the index variables of various factors to find the best solubility of the secretion of the input material (taking the sample to check the solubility).
Fifth—Adjust RPM (Revolutions per minute) of Stirred tanks or reactors or speed of shakers.

There are so many variables, and there will be interactive effects between the variables, which will lead to tedious and lengthy experiments (or trial and error) to find the best parameter mode. Therefore, if there is a simulation prediction model (combining mathematics, statistics, algae, and microbiological science), Just do some experiments to establish a basic parameter database, and find out the regularity, repulsion, etc., and develop an evolutionary calculation formula to design the estimated simulation changes and values. That is to say, the operator only needs to operate the numerical value of each variable of the simulation model. You can see the estimated final output value, reducing the number of trial and error.

Use XYZ axis three axes (X, Y, Z) to present the interactive changes of “variable elements”, “output value” and “time”, and draw up a three-dimensional measurement model

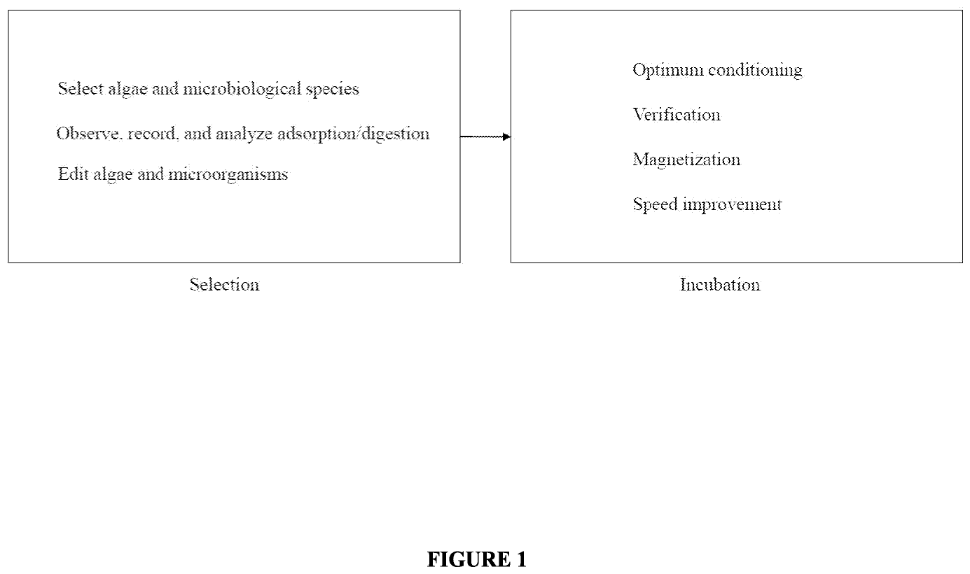

In a particular aspect of the present invention, a screening method is provided that comprises a selection step and an incubation step, as shown in FIG. 1.

The Selection Step:

The selection step may include a process for selecting algae and microbiological species suitable for the recovery of specific heavy metals/REEs from specific input materials. The selection step may include the application of a real-time video electron microscope with which to observe, record, and analyze the performance of algae or microorganism adsorption or digestion.

In some embodiments of the present invention, the step may include the use of genetic engineering to edit particularly suitable algae and microorganisms (e.g., CRISPR-cas12, CRISPR-cas9, CRISPR-cas13, etc.).

The Incubation Step:

The incubation step may include a process for incubating algae and microbiological species to encourage the growth of viable microorganisms or algae. The incubation step may include optimum conditioning, verification, magnetization, and speed improvement.

Optimum Conditioning:

The incubation step may include a process for providing algae or microbiological incubating pool offering optimal growth environment conditions that reflect various factors, such as nutrients, selective agents (allowing certain types of organisms to grow while inhibiting the growth of others), oxygen, temperature, pH, water ability, carbon dioxide, and light (photosynthesis).

In some embodiments of the present invention, paraffin oil can be added to the microbiological incubating pool to coat its surface and avoid direct contact of the contents with air.

Verification:

The incubation step may include a process for verifying the changed volumes of microorganisms and algae. For example, microorganism volumes can be verified through color changes and time calculations.

Magnetization:

Magnetization can be a process for increasing the secretion of a microorganism or intensifying algae growth. For example, by introducing microcurrent or magnetic force in one direction, the electromagnetic field of the algae and microorganisms can be stimulated to boost the algae or microorganism's metabolism, promoting secretion or growth.

The magnetization process may include steps that involve establishing a protective shield against the magnetic field and telecommunications interference from outside the pool, installing and measuring a magnetic field and current counter link to the pool, and installing a secretion/or digestion measurement scale around the pool.

In some embodiments of the present invention, this magnetization step may use the centrifugal principle to separate unnecessary components.

Speed Improvement:

Speed improvement can be a process for improving the speed of secretion dissolution or accelerating algae/microorganism digestion and absorption.

In some embodiments of the present invention, the speed improvement step may use microbial secretions to precipitate specific heavy metal/rare earth elements in the input material. For example, according to the application of the diffusion principle, when the concentration of the secreted solvent exceeds the specific metal concentration of the input material, dissolving specific metals into the secretion solvent is difficult, whereas high concentrations of specific medium metal elements can penetrate a low concentration of the secreted solvent. Thus, specific heavy metal/rare earth elements can be precipitated by taking into account elements of the input traits, such as ionic strength (a measurement of ion concentration in solution) and polarity.

In some other embodiments of the present invention, the speed improvement step may use algae to precipitate specific heavy metal/rare earth elements in the input material. For example, the input material can be absorbed by algae, which may then be collected for drying and heating, with the required heavy metal/rare earth elements obtained by centrifugal separation.

In some embodiments of the present invention, the speed improvement step may include a process for adjusting and controlling various factors, such as pH, temperature, pressure (gas), sunlight, solvent, and salinity.

In some embodiments of the present invention, when necessary, the incubation step of the present invention may further include a mechanical stirring process that uses tanks or reactors for bioleaching or absorption of input materials and to accelerate secretion dissolution or enhance algae's digestion and absorption ability.

In some embodiments of the present invention, the screening method of the present invention can be used with a system that is configured to manage and analyze data relevant to the present invention. Such a system may include a data center and a control center.

The data center may collect real-time information on the analysis of specific microbial concentration, secretion concentration, solubility, and so forth in the microbial culture tank or growth and absorption in the algae incubator.

In some embodiments of the present invention, the data center may also be designed to monitor levels of water, oxygen, carbon dioxide, nutrients, selective agents, temperature, pH, light sources, microcurrent, magnetic field, and any other related information, then send those data to the control center, which may perform automatic analysis and calculation relating to the incubation pool, simulate optimal recovery rate effects, and issue various action commands.

In an embodiment of the present invention, the screening method can be implemented as follows: the user designs a microbial culture tank or algae incubator (“POOL A”) (with various configurations and structural components, such as a water pipe, oxygen tube, carbon dioxide pipe, and nutrients tube); inputs selective agents and adjusts pH, light sources, temperature, and the like; performs sampling port settings (for sampling and analysis of microbe or algae reproduction); and connects POOL A to the data center, which will take current microbe or algae reproduction parameters as the baseline and run computer simulations to calculate optimal adjustments based on various factors (water ability, carbon dioxide, oxygen, nutrients, selective agents) and microbe varieties to produce optimal environmental conditions for POOL A.

For estimated microbial volumes, the data center may perform semiquantitative analysis of microbial content based on color changes and estimate algae volumes by volume and weight scaler, integrating the color change method with ELISA, gene probe, and plate counting methods for higher specificity (99%) and sensitivity (up to 1 CFU/mL), producing rapid test response (5 times faster than traditional methods) through what is called the MBS® method.

The user may wait for specific microorganisms or algae to increase to a certain level in POOL A and add the appropriate amount of milled input materials, simultaneously introducing microcurrent or magnetic force to stimulate the metabolism of microorganisms or algae and boost secretion or absorption percentage. POOL A can be equipped with a protective shield to block the magnetic field and telecommunications interference. The user may adjust sampling port settings further (for sampling and analysis of microbial secretion or algae absorption) and connect POOL A to the data center for the analysis of microbial secretion or algae absorption parameters as the baseline, then perform further computer simulation calculations and calculate various factors (microcurrent or magnetic strength, etc.), updating recommendations for achieving optimal secretion or absorption. The data center may then produce the most suitable conditions for specific microbial or algae metabolic function by creating favorable secretion concentration conditions or absorption levels.

In POOL A, specific microorganisms or algae can be propagated and secreted to a certain concentration, with a significant quantity of a “preparation of selected heavy metal/rare earth input materials” introduced to promote decomposition and dissolution or absorption operations.

The optimal decomposition and melting speed-related parameters estimated by the data center can be provided to the control center, with suitable adjustments made to various factors, such as pH, temperature, and pressure.

In some embodiments of the present invention, the user may perform moderate stirring of POOL A to catalyze precipitation of specific heavy metals/rare earth elements.

The screening method of the present invention may save tens of thousands to millions of dollars in electricity, water, or chemical reagents per day. The present invention also does not create environmental pollution, such as through water or air pollution. The present invention does not produce sulfide, cyanide, or similar residues that would pose workplace health hazards. Furthermore, related algae and microbial vectors can be repeatedly and automatically generated.

The invention will be further explained by the following Examples, which are intended to purely exemplary of the invention, and should not be considered as limiting the invention in any way.

### EXAMPLES

### Example 1—Microbial/Algae Growth Model

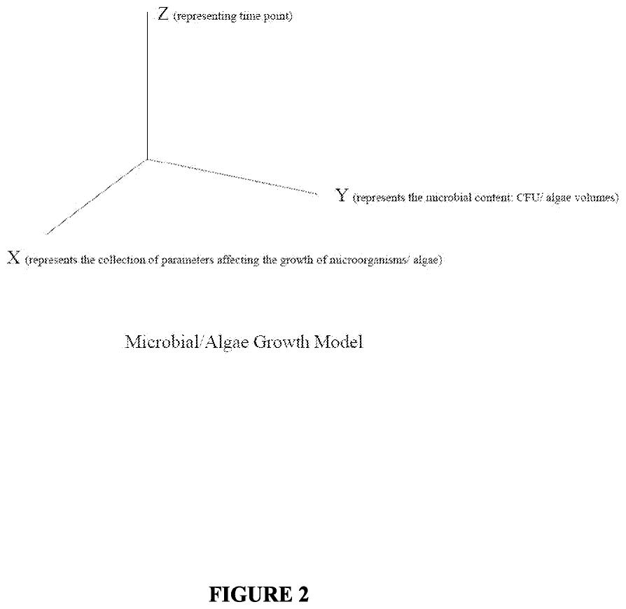

In the microbial/algae growth model, as shown in FIG. 2, where X represents a collection of parameters that affect microorganism/algae growth. These factors that affect the growth of microorganisms/algae are divided into external factors such as temperature, light, pH values, oxygen, carbon dioxide, water ability; and internal factors such as nutrients, selective agent, etc.

The model first fixes the time point and observes the changes in the parameters of various factors that affect the growth of microorganisms, and how the microbial content CFU will change:

a) First to fix each parameter index, and then make different adjustments to the single factor index to obtain the microbial content CFU value record.
For example: in addition to temperature, fixed other external and internal factors, adjust the temperature parameter, and record the microbial content CFU change, find the best temperature point suitable for the growth of the microorganism. And so on, replace the changing factors, and find the best factor index point one by one.
b) It is expanded to two-factor index changes, other factor indexes are fixed, record the change value of microbial content CFU, and find the best combination factor parameter suitable for the growth of the microorganism.
c) Expanded again into three-factor index changes, other factor indexes are fixed, record the change of microbial content CFU, and find the best combination factor parameter suitable for the growth of the microorganism.
d) By analogy, overlap all the icons, observe the differences, and deduce the microbial inertia.

The microbial/algae growth model fixes the time point, tracks changes in the parameters of various factors that affect microorganism/algae growth and predicts changes in microbial content CFU/algae volumes. Each parameter index can first be fixed, with adjustments then made to the single factor index to obtain the microbial content CFU value record/or algae volumes and find the temperature point suitable for, for example, microorganism/algae growth. The user can continue assessing and adjusting factors to find the best index point for each.

In some embodiments of the present invention, the microbial/algae growth model can be expanded to consider two-factor index changes, with other factor indexes fixed and the user recording changes to microbial content CFU/or algae volumes and identifying the combination of factors and parameters most suitable for microorganism/algae growth.

In some embodiments of the present invention, the microbial/algae growth model can be expanded again to consider three-factor index changes, with other factor indexes fixed and the user recording changes to microbial content CFU/or algae volumes and identifying the combination of factors and parameters most suitable for microorganism/algae growth.

Using the microbial/algae growth model, the user may observe differences and deduce microbial/algae inertia.

### Example 2—Microbial/Algae Secretion Model

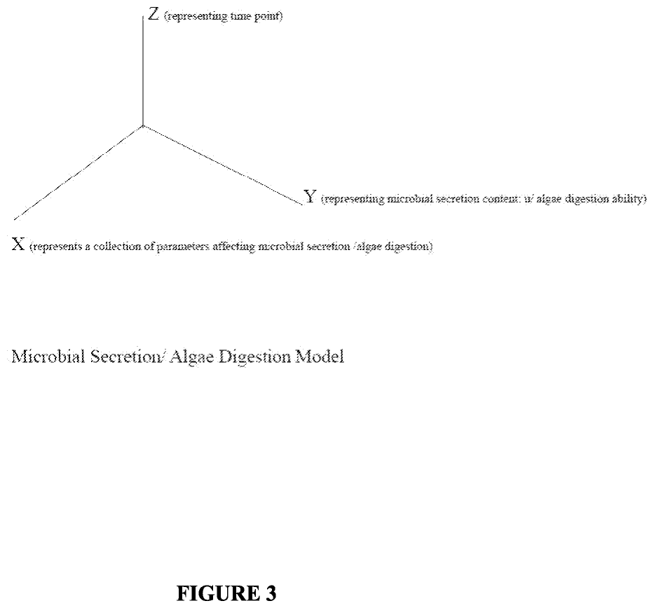

Here, to stimulate the metabolism of microorganisms/algae microcurrent is used or magnetic force is used to increase secretion. In other words, in the microbial secretion/algae digestion model, as shown in FIG. 3, which uses the same XYZ axes, with X representing a collection of parameters that affect microbial secretion/or algae digestion, the user may observe factors related to the stimulation of microorganism/algae metabolism using microcurrent or magnetic force, to boost secretion/or digestion.

### Example 3—Microbial Dissolution Model

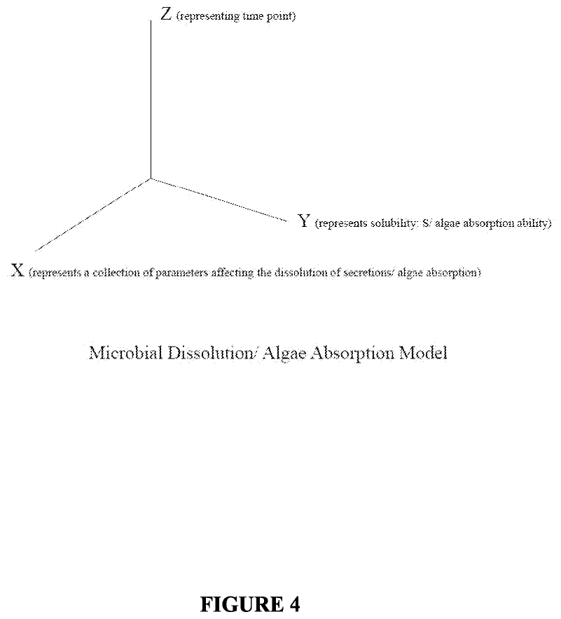

In the microbial dissolution/algae absorption model, as shown in FIG. 4, where X represents a collection of parameters that affect the dissolution of secretion/or absorption and Y represents solubility, the factors that affect the dissolution of secretions/or algae absorption can be divided into external (temperature, pH) and internal factors (ionic strength, polarity). In some embodiments, this model may include a process for mechanically stirring tanks or reactors.

### Example 4—Recovery Rate Measurement Model

The screening method of the present invention can not only increase the recovery rate of related specific elements and reduce impurities but also boost income by 2% to 200% while significantly reducing the steep cost of initial construction of a screening base (with, for example, required land area reduced by two-thirds, eliminating the need to invest in flotation equipment, a gravity table, and so forth).

Many variables are relevant to the present invention, with interactions among them requiring tedious and lengthy experiments (or trial and error) to find ideal parameters. Accordingly, using a simulation prediction model that combines mathematics, statistics, and algae and microbiological science, users can perform experiments to establish a basic parameter database through which to gain insights into regularity, repulsion, and the like, then develop an evolutionary calculation formula for estimating simulation changes and values.

In short, the user may need only adjust the numerical value of each variable of the simulation prediction model to monitor the estimated final output value, reducing reliance on trial and error.

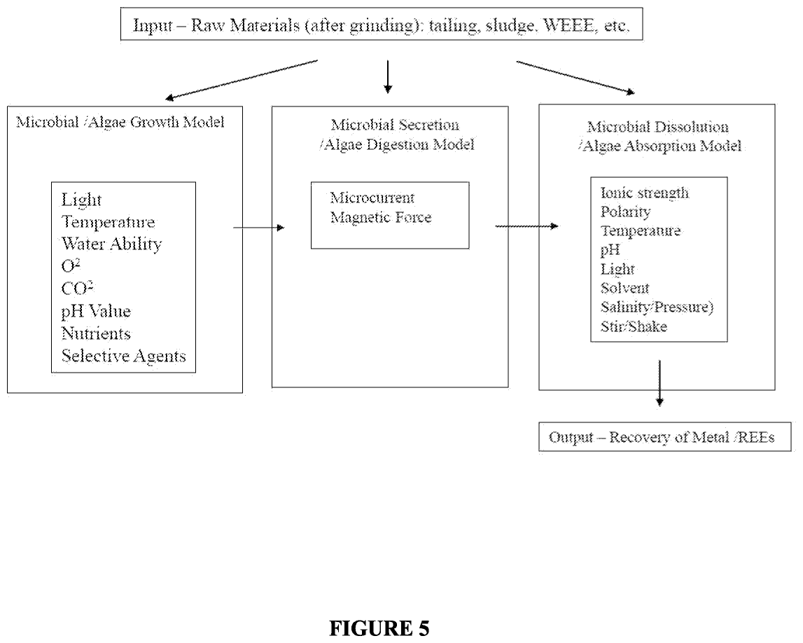

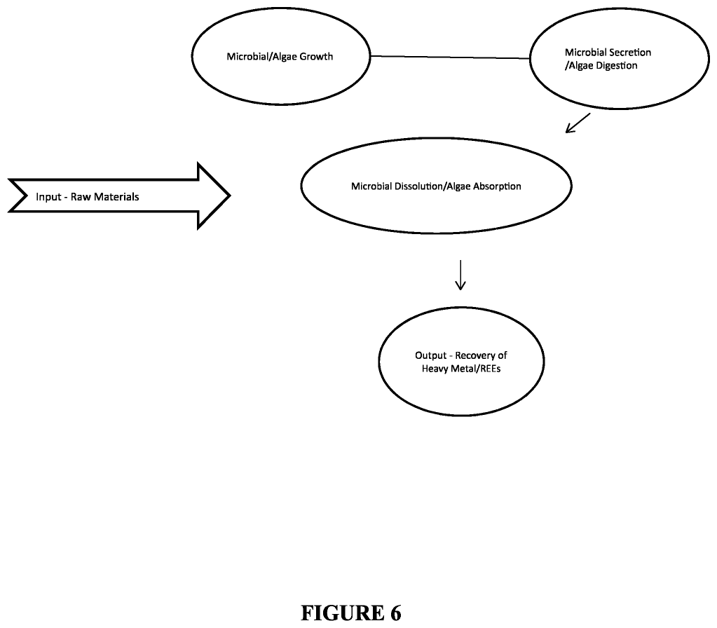

For example, the user may use three axes (X, Y, Z) to plot interactive changes in “variable elements,” “output value,” and “time,” using them to create a three-dimensional measurement model (simulation prediction model) such as a microbial/algae growth model, microbial secretion/algae digestion model, and microbial dissolution/algae absorption model, which can be sequentially combined to represent the general process of the present invention, as shown in FIGS. 5 and 6.

In some embodiments, the data center may include a recovery rate metric model (RRM) that may be configured to predict recovery rate, then let the control center adjust factors to promote recovery of heavy metals/REEs using microbiology or algae.

The RRM may include one or more stochastic equations to reveal the quantitative relationship between various factors in the screening activities.

In the RRM model, a relationship or an equation can be generated. An exemplar relationship can be as following: Input>Total microbial content (CFU)×Average secretion of one unit of microorganism (u)×Solubility (S)>Output as shown in FIGS. 5 and 6.

To summarize, the present invention is advantageous and technically advanced over the other known conventional and traditional methods and systems for screening of heavy metals and/or REEs in terms of:

1. The present method will save 9/10 of energy/water consumption.
2. No consumables or chemical agents requested.
3. Manufacturers may save tens of thousands to millions of dollars in daily electricity, water, or chemical reagents.
4. Safety for labor Health
5. No pollution problems, such as wastewater or air pollution, and more. No health hazard concerns about sulfide, cyanide residues, etc.
6. Related microbial vectors can be automatically generated repeatedly.
7. Increase the recovery rate of specific elements and reduce impurity content, it can generate 2% to 200% increased income. Reduce the initial screening base construction cost (for example, the required land area is reduced by 2/3, eliminating investment in flotation equipment and gravity table).
8. Suitable for the screening and purification of low-grade mines, tailings, rare earth, silt, and specific elements of Waste Electrical and Electronic Equipment (WEEE).

Also, the disclosure according to the present invention provides a green tech method and design which is applied for the number of input materials to stimulate the screening speed of algae & microorganisms. The target customers are waste electrical and electronic equipment recycling industry, miner or ICT hardware manufacturing industry that produces industrial sludge. If there are adequate input materials (for example, tailings, mines, sludge containing heavy metal elements and waste electronic and electrical equipment), depending on the value of the element content of the input materials, usually, once the installation of the facility is completed and the operation starts, it only takes 6 months to 1 year to see and receive the investment payback.

It will be apparent to those skilled in the art that various modifications and variations can be made in the practice of the present invention without departing from the scope or spirit of the invention. Other embodiments of the invention will be apparent to those skilled in the art from considering of the specification and practice of the invention. It is intended that the specification and examples be considered as exemplary only, with a true scope and spirit of the invention being indicated by the following claims.

1. A method of algae and microbial screening for recovery of specific heavy metals and/or rare earth elements (REEs), the method comprising the steps of:
selecting specific algae or microbial species by screening for specific heavy metals or REEs to find a specific algae or microbial species and analyzing the optimal size of input materials for the adsorption or repulsion effect on the input material by the specific algae or microbial species;
adjusting the index variables of various factors in an incubation pool referred to as Pool A to find the best growth conditions for the specific algae or microbial species involving collecting a sample from a sampling port for sampling and analysis by an information data and control center for the reproduction of the specific algae and microbial species by testing the sample for concentration or growth;
modulating the environmental conditions in the incubation pool by adjusting micro-current or magnetic variables to stimulate algae or microbial species metabolism and increase secretion or absorption and verifying it by collecting a sample from the sampling port for sampling and analysis by the information data and control center;
readjusting the index variables of various factors in the incubation pool to find the best solubility of the secretion of the input material and verifying it by collecting a sample from the sampling port for sampling and analysis by the information data and control center; and
adjusting the revolutions per minute referred to as rpm of stirred tanks or reactors or speed of shakers in the incubation pool in an interactive manner by coordinating through the information data and control center.

2. The method of claim 1, wherein the adsorption or repulsion effect on the input material by the specific algae or microbial species consists of:
(i) fully grinding, diluting and decomposing the excitation viable algae and microbial species to obtain secretions from specific excitation viable algae and microbial species referred to as (A) which decompose specific heavy metals and/or REEs into ions and precipitate the specific heavy metals and/or REEs referred to as (X), or
(ii) fully grinding, diluting and decomposing the excitation viable algae and microbial species to obtain secretions from specific excitation viable algae and microbial species referred to as (B) which repel specific heavy metals and/or REEs and produce the precipitation of the heavy metal and/or REEs referred to as (Y).

3. The method of claim 1, wherein the various factors consist of external and internal factors, wherein the external factors comprise temperature, light, pH value, oxygen, carbon dioxide, amount of water, and wherein the internal factors comprise nutrients, selective agents, ionic strength, polarity.

4. The method of claim 1, wherein the information data and control center can adjust the index variables of various factors in the incubation pool to shift the mode of operation of the incubation pool in an outcome selected from a group consisting of growth mode, secretion mode, dissolution mode, recovery rate measurement mode, or a combination thereof, for recovery of specific heavy metals and/or REEs.

5. A method for improving the speed of algae and microbial screening for recovery of specific heavy metals and/or rare earth elements (REEs), the method comprising the steps of:
selecting specific algae and microbial species for specific heavy metal and/or REE and input materials;
incubating said specific algae and microbial species in an incubation pool referred to as Pool A, comprising specific nutrients and selective agents to stimulate excitation viable algae and microbial species that show rapid growth;
verifying the change in volume as a measure of said rapid growth of said excitation viable algae and microbial species;
modulating the environmental conditions in the incubation pool to obtain specific excitation viable algae and microbial species;
adding milled input materials to the incubation pool;
recovering specific heavy metals and/or REEs from said input materials by algae and microbial screening using the excitation viable algae and microbial species by selecting from a group consisting of (i) fully grinding, diluting and decomposing the excitation viable algae and microbial species to obtain secretions from specific excitation viable algae and microbial species referred to as (A) which decompose specific heavy metals and/or REEs into ions and precipitate the specific heavy metals and/or REEs referred to as (X), or (ii) fully grinding, diluting and decomposing the excitation viable algae and microbial species to obtain secretions from specific excitation viable algae and microbial species referred to as (B) which repel specific heavy metals and/or REEs and produce the precipitation of the heavy metal and/or REEs referred to as (Y), or (iii) using specific excitation viable algae to precipitate specific heavy metals and/or REEs present in the input materials after being absorbed by algae and collecting said algae for drying and heating, and obtaining the specific heavy metals and/or REEs by centrifugal separation, or a combination thereof;
sampling and monitoring continuously the specific excitation viable algae and microbial species by collecting samples from a sampling port; and
analyzing the collected samples for parameters of microbial secretion or algae absorption and modulating the environmental conditions in the incubation pool by an information data and control center by the application of a recovery rate metric model referred to as RRM to identify and select the most suitable specific algae and microbial species for a specific heavy metals and/or REEs in the input materials and for improving the speed of algae and microbial screening for recovery of said specific heavy metals and REEs.

6. The method of claim 5, wherein the modulating the environmental conditions in the incubation pool to obtain specific excitation viable algae and microbial species results from selecting a mode of operation from a group consisting of intensifying algae growth or increasing the secretion of secretions from the microbial species or improving the speed of secretion dissolution of microbial species or enhancing the speed of algae digestion and absorption, or a combination thereof.

7. The method of claim 5, wherein the application of a recovery rate metric model referred to as RRM comprises recovery rate prediction, and adjusting various factors suitably for identification and selection of the most suitable specific algae and microbial species for a specific heavy metals and/or REEs in the input materials and for improving the speed of algae and microbial screening for recovery of said specific heavy metals and REEs, wherein the various factors consist of external and internal factors,
wherein the external factors comprise temperature, light, pH value, oxygen, carbon dioxide, amount of water, and
wherein the internal factors comprise nutrients, selective agents, ionic strength, polarity.

8. The method of claim 5, wherein the recovery rate metric model referred to as RRM is integrated into intelligent evolutionary learning platform involving machine learning run by the information data and control center and comprises one or more stochastic equations which are composed of variables and coefficients to identify and select the most suitable specific algae and microbial species for a specific heavy metals and/or REEs in the input materials and for improving the speed of algae and microbial screening for recovery of said specific heavy metals and REEs, wherein a set of minimums (m) most suitable marked algae or microbial species are identified along with influencing factors from high-dimensional (n) training samples to establish a mathematical prediction model.

9. A system for the method for improving the speed of algae and microbial screening for recovery of specific heavy metals and rare earth elements (REEs), the system comprising:
an incubation pool used as a microbial culture tank or algae incubator referred to as Pool A;
a sampling port for sampling and analysis of microbial species and algae reproduction; and
an information data and control center,
wherein, the information data and control center comprises: collecting real-time information on the analysis of specific parameters comprising microbial concentration, secretion concentration, solubility of the microbial culture tank, the growth of microbial species and absorption of algae incubator; monitoring the amount of water, oxygen, carbon dioxide, nutrients, selective agents, temperature, pH value, light sources strength, micro-current, magnetic field, and sending the collected and monitored information to the control center, analyzing automatically and calculating the optimal recovery rate effect simulation for Pool A, and issuing the various ACTION commands for Pool A for said optimal recovery rate effect by the application of a recovery rate metric model referred to as RRM to identify and select the most suitable specific algae and microbial species for a specific heavy metals and/or REEs in the input materials and for improving the speed of algae and microbial screening for recovery of said specific heavy metals and REEs, and
wherein the sampling port is connected to the information data and control center.
